(19) United States
(12) Patent Application Publication
(10) Pub. No.: US 2005/0130275 A1
(43) Pub. Date: Jun. 16, 2005

(54) Process for preparing enzymatically debranched starch and application thereof

(75) Inventors: Shyh-Yang Lee, Hsinchu city (TW); Ming-Yi Chu, Hsinchu City (TW); Wen-Faa Kuo, Hsinchu City (TW); Chia-Hao Hu, Taipei County (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsin Chu (TW)

(21) Appl. No.: 10/890,272

(22) Filed: Jul. 14, 2004

(30) Foreign Application Priority Data
Dec. 12, 2003 (TW) 92134825

(51) Int. Cl. C12P 19/04; C08B 31/00

(52) U.S. Cl. 435/101; 536/102

(57) ABSTRACT

The present invention relates to a process for preparing enzymatically debranched starch by mixing natural starch, water and polyalcohol to form a starch mixture, adding α-1,6-D-glucanohydrolase to debranch the natural starch under 50˜70° C., and terminating the debranching reaction by increasing the temperature of starch mixture. The process of this invention can achieve the purpose of degrading starch to lower molecular weight, while raising amylose content of starch so that the plasticity of starch is increased.

### BACKGROUND OF THE INVENTION

1. Field of the Invention

The present invention discloses a process for preparing enzymatically debranched starch, in which enzymatic engineering is utilized to carry out structural sectioning and control of starch molecules, the granules of natural starch miniaturized, linear converted and the molecular weight lowered, and thereby raise the content of amylose and enhance the plasticity of starch.

2. Description of Related Art

Starch molecules are found as granules in plant cells. The sizes of the granules range from 30˜100 μm, depending on the crops. The starch molecules in the granule are compactly arranged due to the abundance of hydrogen bonds and the double helical lattice structure, which make the destructuring and processing of starch difficult. Natural starch polymers typically contain linear starch amylose (about 25%) and branched starch amylopectin (about 75%), where only amylose provides superior mechanical property due to the structural characteristics. Besides adding directly or use denatured starch by modifying natural starch, the major applications of starch are adding the natural starch as low molecular weight monosaccharides or polysaccharides by various means of destructuring.

U.S. Pat. No. 4,971,723 discloses a process for preparing partially debranched starch by enzymatic hydrolysis, which changes the structure of amylopectin in starch and increases the content of short-chain amylose to 80%. Said process comprises the steps of gelatinizing starch slurry (20-30% solid), lowering the slurry to 58-60° C., and then adding α-1,6-D-glucanohydrolase to debranch the starch after adjusting the pH value. U.S. Pat. Nos. 3,730,840, 3,881,991, 3,879,212, and 4,971,723 all use similar process to debranch the starch.

Due to heightened awareness to environmental protection in recent years, it is an inexorable trend in polymer industry to develop biodegradable plastic materials as an alternation of the commodity, non-degradable plastics, such as PP and PE. Therefore, using starch as a biodegradable plastic material has been the focus of recent research endeavors. It is well known that only amylose offers better mechanical property, however, the processes for debranching starch disclosed in the aforementioned US patents involve many steps and lengthy reaction time of over 24 hours. Most important of all, the drying process will be an inevitable issue in the manufacture of plastics owing to the slurry state of the debranched starch. Consequently, there is still much room for improvement of those processes for the expectancy of applying such starch in the mass production of biodegradable plastic materials.

Accordingly, the present invention discloses a new process for debranching starch, which greatly shortens the process time and effectively controls the molecular weight of starch to achieve the miniaturization, linear conversion and lower molecular weight of starch granules and thereby raise the content of amylose and enhance the plasticity of starch.

### SUMMARY OF THE INVENTION

The present invention relates to a process for preparing enzymatically debranching starch, comprising the steps of:

(a) mixing natural starch, water and polyalcohol in the ratio of 100 parts of starch by weight, 10-40 parts of water by weight, and 5-40 parts of polyalcohol by weight to form a starch mixture;

(b) adding α-1,6-glucanohydrolase to the starch mixture in step (a) in the amount of 5-60 U per gram of starch to undergo debranching reaction under 50˜70° C.; and

(c) terminating the aforesaid enzymatic debranching reaction to obtain enzymatically debranched starch.

The aforesaid polyalcohol includes glycerin, erythritol, pentitol and hexito, preferably glycerin.

The temperature of debranching reaction in step (b) is preferably 60° C.

The duration of debranching reaction in step (b) may be adjusted depending on needs; the longer the reaction time, the higher the degree of debranching is obtained.

The termination of enzymatic debranching reaction in step (c) may be achieved by placing the starch mixture under 85˜110° C., preferably 90° C., for at least 10 minutes.

The aforesaid natural starch comprise corn starch, potato starch, wheat starch, tapioca starch, or waxy corn starch.

The aforesaid α-1,6-glucanohydrolase comprises pullulanase or α-isoamylase.

The aforesaid natural starch becomes 10˜90% debranched after the enzymatic debranching reaction.

In the aforesaid process for preparing enzymatically debranched starch, the debranching reaction in step (b) can take place in a kneader.

The mixture of natural starch, water, and polyalcohol in the starch mixture is preferably 100 parts of starch by weight, 10˜40 parts of water by weight, and 5˜20 parts of polyalcohol by weight.

The present invention further provides an enzymatically debranched starch prepared according to the process described above; said enzymatically debranched starch comprises amylose, amylopectin and polyalcohol, is 10˜90% debranched.

The aforesaid enzymatically debranched starch is natural starch, comprising corn starch, potato starch, wheat starch, tapioca starch, or waxy corn starch.

The aforesaid α-1,6-glucanohydrolase comprises pullulanase or α-isoamylase.

The aforesaid enzymatically debranched starch can be further mixed with natural starch, if desired, and used as plastic material; the aforesaid polyalcohol includes glycerin, erythritol, pentitol and hexito, preferably glycerin.

Another purpose of the present invention is to provide a starch-based plastic material, comprising (a) 10˜100 wt % of said enzymatically debranched starch prepared according to the aforesaid process; and (b) 0˜90 wt % of natural starch.

The aforesaid enzymatically debranched starch comprises amylose, amylopectin and polyalcohol, wherein the total weight of amylose and amylopectin is 100 parts by weight, the content of polyalcohol is 30˜40 parts by weight.

The aforesaid α-1,6-glucanohydrolase comprises pullulanase or α-isoamylase.

The aforesaid enzymatically debranched starch comprises amylose, amylopectin and polyalcohol, and is 10˜90% debranched.

The aforesaid plastic material preferably comprises (a) 50˜90 wt % of aforesaid enzymatically debranched starch prepared according to the aforesaid process; and (b) 10˜50 wt % of natural starch.

The aforesaid enzymatically debranched starch is 40˜60% debranched.

The aforesaid enzymatically debranched starch is natural starch, comprising corn starch, potato starch, wheat starch, tapioca starch, and waxy corn starch.

This invention provides a new process for enzymatically debranched starch, in which the chain length and the branch structure of the starch may be controlled. The process disclosed in this invention may be developed into starch technology specifically for industrial applications. The enzymatically debranched starch acquired thereof possesses stable microstructure and displays the characteristics of polymer via plasticity control technology, and may be applied in the development of 100% starch-based biodegradable plastic, hydrophilic polymer resin, industrial-grade packing buffer, and oxygen-resistant, water-penetratable packing material. These applications represent breakthrough of existing technologies. In the future, plastic materials may be produced from fast-growing plants, which signifies a giant step in environmental protection. Also, the low costs of industrial starch can greatly reduce the prices of plastic products, reversing the persistent trend of high manufacturing and processing costs of biodegradable polymers at the present time.

### BRIEF DESCRIPTION OF THE DRAWINGS

FIG. 1 depicts the flow chart of the process for preparing enzymatically debranched starch according to the present invention.

### DETAILED DESCRIPTION OF THE INVENTION

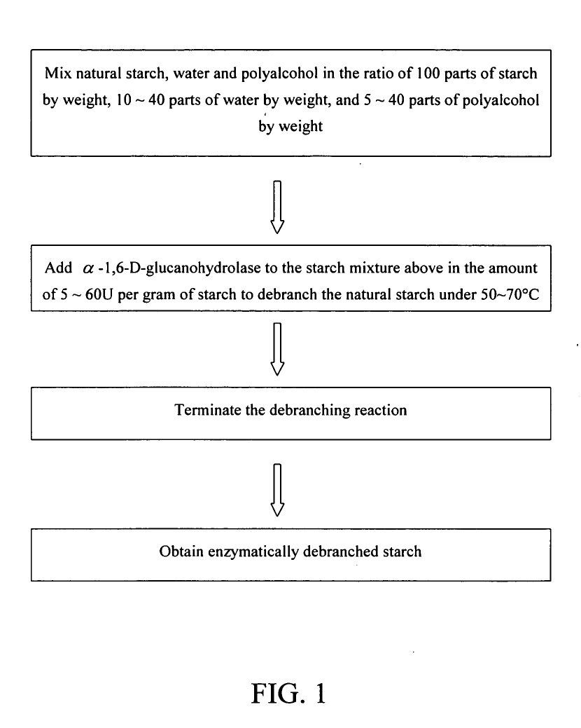

The present invention provides a process for preparing enzymatically debranched starch as shown in FIG. 1, comprising the following steps: first mix natural starch, water and polyalcohol in the ratio of 100 parts of starch by weight, 10˜40 parts of water by weight, and 5˜40 parts of polyalcohol by weight, preferably 5˜20 parts of polyalcohol, which may be glycerin, erythritol, pentitol or hexito, preferably glycerin. Next, add α-1,6-glucanohydrolase to the starch mixture in the amount of 5-60 U per gram of starch to conduct debranching reaction under 50˜70° C., preferably at 60° C.; the duration of reaction time may be adjusted based on the desired percentage of debranching in the starch; place the aforesaid starch mixture under 85˜110° C., preferably at 90° C., for at least 10 minutes to terminate the debranching reaction to obtain enzymatically debranched starch that is 10˜90% debranched.

The aforesaid natural starch comprises corn starch, potato starch, wheat starch, tapioca starch, or waxy corn starch.

The aforesaid α-1,6-glucanohydrolase comprises pullulanase or α-isoamylase.

In the aforesaid process for preparing enzymatically debranched starch, the debranching reaction can take place in a kneader.

The aforesaid enzymatically debranched starch can be further mixed with natural starch and used as plastic material, if desired, wherein the mixing ratio is 10˜100 wt % of enzymatically debranched starch and (b) 0˜90 wt % of natural starch, preferably 50˜90 wt % of enzymatically debranched starch and 10˜50 wt % of natural starch. The polyalcohol, used in the process of the enzymatically debranched starch as a component of plastic material, contains at least 30 parts by weight, while the starch is 100 parts by weight.

The advantages of the present invention are further depicted with the illustration of examples, but the descriptions made in the examples should not be construed as a limitation on the actual application of the present invention.

### EXAMPLE 1

### Preparation of Enzymatically Debranched Starch

Mix starch, water, glycerin and pullulanase in the ratio as shown in Table 1 below, in which SED-70, SED-71, and SED-72 are debranched corn starches, and SED-76 is potato starch. The addition of pullulanase is 20 U per gram of starch. Glycerin and water are added as plasticizer, which will make the penetration of enzyme into the starch granules easier to produce structural degradation of the molecules.

Place the mixture of starch, water, glycerin and pullulanase in a kneader to knead the mixture well which will help enzymatic control over the sectioning of starch molecules, thereby raising the content of amylose, and control over the stability of microstructure, thereby enhancing the plasticity of starch; control the temperature of debranching reaction at 60° C. Let the reaction go on for six hours, and sample at the interval of one hour apart throughout the reaction and place the sample in ethanol to deactivate the enzyme. Afterwards, remove the sample from ethanol and dry it, and then measure the inherent viscosity (IV) of starch to learn the degree of starch degradation over time. The conditions for measuring inherent viscosity are: 0.5 wt % of starch, solvent: DMSO/H₂O(90/10), and measuring temperature: 30° C.

TABLE 1
Variation of IV values of starches with reaction time undergoing
enzymatic debranching

| Sample Name | SED-70 | SED-71 | SED-72 | SED-76 |
|---|---|---|---|---|
| Corn starch (g) | 100 | 100 | 100 | 0 |
| Potato starch | 0 | 0 | 0 | 100 |
| Glycerin (phr) | 20 | 10 | 0 | 10 |
| Water (phr) | 20 | 30 | 40 | 30 |
| Pullulanase Enzyme (U/g) | 20 | 20 | 20 | 20 |
| Debranched temp (° C.) | 60 | 60 | 60 | 60 |
| Inherent viscosity (dl/g) | | | | |
| IV at 0 H | 1.74 | 1.74 | 1.74 | 3.42 |
| IV at 0.5 H | 1.386 | 1.366 | 1.358 | 2.34 |
| IV at 1 H | 1.351 | 1.274 | 1.218 | 2.14 |
| IV at 2 H | 1.305 | 1.140 | 1.112 | 1.60 |
| IV at 3 H | 1.218 | 1.072 | 1.095 | 1.75 |
| IV at 4 H | 1.244 | 1.018 | 1.108 | 1.50 |
| IV at 5 H | 1.261 | 0.918 | 1.148 | 1.64 |
| IV at 6 H | 1.279 | 0.856 | 1.131 | 1.55 |

*phr (parts per hundred ratio) means the parts of other ingredients by weight added when starch is 100 parts by weight.

Table 1 depicts the variation of IV values of starches with reaction time undergoing enzymatic debranching. As shown in Table 1, the IV values of the starches reduce significantly over time. The IV value of the corn starch (SED-71) dropped from 1.74 to 0.85 after six hours of reaction. For SED-76 that had the same composition as SED-71 except that potato starch was used instead the IV value dropped from 3.42 to 1.50 after enzymatic debranching.

### EXAMPLE 2

### Analyzing the Degree of Debranching in Enzymatically Debranched Starch

Starch samples debranched for three and six hours were subjected to HPSEC (High Performance Size Exclusion Chromatography) examination to learn about the molecular structure of starches and the results are given in Table 2.

TABLE 2
Degree of Debranching of Amylopectin

| | Amylopectin | | |
|---|---|---|---|
| | Ave. No. | Ave No. | Degree of |
| Starch | of units | of branches | debranching (%) |
| Native corn starch | 378,148 | 18,007 | — |
| SED-70-3H | 218,457 | 9,930 | 44.9 |
| SED-70-6H | 214,938 | 9,770 | 45.7 |
| SED-71-3H | 184,383 | 8,780 | 51.2 |
| SED-71-6H | 161,667 | 7,698 | 57.2 |
| SED-72-3H | 177,407 | 8,064 | 55.2 |
| SED-72-6H | 165,062 | 8,253 | 54.2 |

Degree of debranching = (Original average number of branches of starch − average number of branches after degradation)/original average number of branches

Table 2 depicts the degree of debranching of enzymatically debranched corn starch. As shown in Table 2, the average number of branches of the native corn starch was 18,007. The number of its branches dropped significantly after 3˜6 hours of enzymatic debranching, down to the level of 9,930˜7,698. The degree of debranching was 45.7% in SED-70-6H, 57.2% in SED-71-6H, and 54.2% in SED-72-6H. The results indicate that Pullulanase can convert amylopectin into linear chain, and thereby increases the content of amylose in starch.

### EXAMPLE 3

### Preparation of Starch-Based Plastic Material

Prepare SED-76 in Example 1 by mixing potato starch, water, glycerin and enzyme in a kneader and let the reaction go on for six hours at 60° C., and then add 20 phr of glycerin to obtain DP/SED-76 (enzymatically debranched starch); remove the debranched starch mixture from kneader and place it in oven at 90° C. for 2 hours to deactivate the enzyme and dry. Afterwards, crush the starch into fine powder to obtain the structurally-controlled debranched starch. In addition, mix and place 100 parts of native potato starch by weight and 30 phr glycerin in kneader for 1 hour; remove the resulting starch mixture from the kneader and place it in oven at 90° C. for 2 hours to dry; crush the starch into fine powder to obtain starch NP/SED-80 (non-debranched starch).

Prepare NP/SED-80 and DP/SED-76 in varying compositions (Samples 1˜7 in Table 3) and pass them through single-screw extruder to turn out plastic pellets. By comparing the processibility of the starches that underwent enzymatic debranching (DP-SED-76, Samples 2˜7) and of the starch that was not subject to enzymatic debranching (NP/SED-76, Sample 1), it is found as shown in Table 3 that the processing temperature required for Samples 2˜7 throughout the extrusion process was significantly lower than that for Sample 1. The same phenomena were observed for the melting temperature of the processed starch, while all processed starches were made into pellets under the setting processing conditions.

TABLE 3
Comparison of Processing Temperature for the Plasticization of Starch

| Sample | 1 | 2 | 3 | 4 | 5 | 6 | 7 |
|---|---|---|---|---|---|---|---|
| NP/30 phr G | 0 | 100 | 300 | 500 | 700 | 900 | 1000 |
| (SED-80) | | | | | | | |
| DP/30 phr G | 1000 | 900 | 700 | 500 | 300 | 100 | 0 |
| (SED-76) | | | | | | | |
| Drying temp | 90° C., 2 hours | | | | | | |
| Extruder temp | Φ = 25 mm, L/D = 28, screw speed = 20 rpm | | | | | | |
| Zone 1* ° C. | 120/120 | 120/119 | 120/119 | 120/120 | 125/125 | 130/129 | 150/149 |
| Zone 2* ° C. | 130/131 | 130/131 | 130/130 | 130/131 | 130/132 | 135/137 | 165/167 |
| Zone 3* ° C. | 140/141 | 140/141 | 140/141 | 140/141 | 140/140 | 140/141 | 175/174 |
| Zone 4* ° C. | 135/134 | 135/138 | 136/137 | 135/138 | 135/138 | 135/138 | 175/176 |
| Melting temp (° C.) | 141 | 137 | 136 | 140 | 139 | 135 | 175 |
| Die pressure (psi) | 750 | 820 | 900 | 1100 | 1280 | 1420 | 2400 |
| Pelletization | OK | OK | OK | OK | OK | OK | OK |

*Set temp./Real temp.

### EXAMPLE 4

### Application of Starch-Based Plastic Material in the Manufacture of Sheets

Prepare partially debranched starch having IV value between 1.6˜1.8 using potato starch and the debranching process described in Example 1. Prepare starch blends using the aforesaid partially debranched starch and subject them to extrusion according to the mixture ratios and processing conditions depicted in Example 3 to obtain plastic pellets with the size of approximately 3 mm. Use the resulting starch-based plastic pellets to make sheets according to the method described below:

Use hollow, stainless steel moulding frame with outer-rim in the dimensions of 24 cm×24 cm, width of 2 cm and thickness of 1 mm. First spread 70 g of starch pellets evenly in the hollow portion of the frame. Then use vacuum hot press molding machine to apply 800 psi of pressure at 150° C. in vacuum environment continuously for 10 minutes to form the sheet sample that is 20 cm×20 cm with thickness of 1 mm.

Prepare standard tensile bar specimens from the sheet samples made from starch pellets with different compositions shown in table 3, and subject them to tensile test based on the ASTM D638 method to measure the tensile properties of the specimens. The results are summarized in Table 4.

TABLE 4
Tensile properties of the sheet samples made from starch pellets
with different compositions.

| Sample | 1 | 2 | 3 | 4 | 5 | 6 | 7 |
|---|---|---|---|---|---|---|---|
| Tensile strength | 42.3 | 55.5 | 50.0 | 51.1 | 48.2 | 47.1 | 39.6 |
| (Kgf/cm²) | | | | | | | |
| Elongation (%) | 39.5 | 39.2 | 34.0 | 30.0 | 26.3 | 28.5 | 27.1 |

According to the results shown in Table 4, The tensile strength of the sheets made of partially debranched starch (Sample 1) was slightly higher than those made of unmodified starch (Sample 7), while the elongation was much higher. Also, sheets made of the blends of partially debranched starch and unmodified starch (Samples 2 -6) possessed tensile strength superior to that of the specimen made of the unmodified starch (Sample 7). The elongation of the sheet samples exhibited rising trend as the content of debranched starch increased.

### EXAMPLE 5

### Application of Starch Material in the Manufacture of Thin Film

Prepare partially debranched corn starch and potato starch using the enzymatic debranching process described in example 1 to obtain partially debranched starch with IV value of 0.85 and 1.60 respectively. Use the resulting materials to produce thin films as described below.

Spread 10 g of partially debranched starch evenly in 90 g of distilled water and then conduct gelatinization under proper temperature for a period of time as shown in Table 5 to obtain transparent, viscous, gelatinized starch solution. After the starch solution is cooled to room temperature, use a scraper to remove about 20 g of the gelatinized starch and spread it on a glass plate to form a thin film with an area of 30 cm×20 cm and thickness of 150 μm. Let the thin film stand under ambient temperature for 24 hours to dry and finally obtain a clear and transparent starch film of 21 μm in thickness.

Prepare standard specimens from the starch films and subject them to test according to ASTM D882 method to measure the tensile properties of the specimens. The results are summarized in Table 5.

TABLE 5
The gelatinization conditions used for corn and potato starches and
the respective resulting tensile properties.

| Sample | Corn starch | Potato starch |
|---|---|---|
| Gelatinization temp (° C.) | 88 | 72 |
| Gelatinization time (hr) | 4.0 | 2.5 |
| Tensile strength (Kgf/cm²) | 355 | 416 |
| Elongation (%) | 0.90 | 1.68 |

Other Embodiment

The present inventions may also be applied in the manufacture (applications) of totally biodegradable plastic materials, manufacture (applications) of biofragmentation plastic materials, manufacture (applications) of photo/oxidation/bio-degradable plastic materials, cosmetics/skin care products, paper making, and adhesives and thickeners.

The preferred embodiments of the present invention as disclosed above are not meant to limit this invention. All modifications and alterations made by those familiar with the skill without departing from the spirits of the invention, including other embodiments mentioned above and appended claims shall remain within the protected scope and claims of the invention.

1. A process for preparing enzymatically debranching starch, comprising the steps of:
(a) mixing natural starch, water and polyalcohol in the ratio of 100 parts of starch by weight, 10-40 parts of water by weight, and 5-40 parts of polyalcohol by weight to form a starch mixture;
(b) adding α-1,6-glucanohydrolase to the starch mixture in step (a) in the amount of 5-60 U per gram of starch to undergo debranching reaction under 50˜70° C.; and
(c) terminating said enzymatic debranching reaction to obtain enzymatically debranched starch.

2. The process according to claim 1, wherein said polyalcohol includes glycerin, erythritol, pentitol and hextio.

3. The process according to claim 2, wherein said polyalcohol is glycerin.

4. The process according to claim 1, wherein the temperature of debranching reaction in step (b) is 60° C.

5. The process according to claim 1, wherein the duration of debranching reaction in step (b) can be adjusted according to needs; the longer the reaction time, the higher the degree of debranching is obtained.

6. The process according to claim 1, wherein the termination of enzymatic debranching reaction in step (c) can be achieved by raising and maintaining the reaction temperature to 85˜110° C. for at least 10 minutes.

7. The process according to claim 6, wherein the temperature for terminating enzymatic debranching reaction in step (c) is 90° C.

8. The process according to claim 1, wherein said natural starch comprises corn starch, potato starch, wheat starch, tapioca starch, or waxy corn starch.

9. The process according to claim 1, wherein said α-1,6-glucanohydrolase comprise pullulanase or α-isoamylase.

10. The process according to claim 1, wherein said natural starch is 10˜90% debranched after the enzymatic debranching reaction.

11. The process according to claim 1, wherein said debranching reaction in the process for preparing enzymatically debranched starch can take place in a kneader.

12. The process according to claim 1, wherein the mixing ratio of natural starch, water, and polyalcohol in step (a) is 100 parts of starch by weight, 10˜40 parts of water by weight, and 5˜20 parts of polyalcohol by weight.

13. An enzymatically debranched starch prepared according to the process of claim 1, which comprises amylose, amylopectin and polyalcohol and is 10˜90% debranched.

14. The enzymatically debranched starch according to claim 13, wherein said enzymatically debranched starch is debranched by pullulanase or α-isoamylase.

15. The enzymatically debranched starch according to claim 13, wherein said polyalcohol comprises glycerin, erythritol, pentitol and hexito.

16. The enzymatically debranched starch according to claim 15, wherein said polyalcohol is glycerin.

17. The enzymatically debranched starch according to claim 13, wherein the starch is natural starch, comprising corn starch, potato starch, wheat starch, tapioca starch, or waxy corn starch.

18. The enzymatically debranched starch according to claim 13, wherein said enzymatically debranched starch can be further mixed with natural starch and used as plastic material.

19. A starch-based plastic material, comprising:
(a) 10˜100 wt % of enzymatically debranched starch according to claim 13; and
(b) 0˜90 wt % of natural starch.

20. The starch-based plastic material according to claim 19, wherein said enzymatically debranched starch comprises amylose, amylopectin and polyalcohol, that when the total weight of amylose and amylopectin is 100 parts by weight, the content of polyalcohol is 30˜40 parts by weight.

21. The starch-based plastic material according to claim 19, wherein said enzymatically debranched starch comprises amylose, amylopectin and polyalcohol, and is 10˜90% debranched.

22. The starch-based plastic material according to claim 19, wherein said plastic material comprises:
(a) 50˜90 wt % of enzymatically debranched starch according to claim 13; and
(b) 10˜50 wt % of natural starch.

23. The starch-based plastic material according to claim 19, wherein said enzymatically debranched starch is natural starch, comprising corn starch, potato starch, wheat starch, tapioca starch, or waxy corn starch.

24. The starch-based plastic material according to claim 19, wherein said plastic material can be applied in the manufacture of sheets.

25. The starch-based plastic material according to claim 19, wherein said plastic material can be applied in the manufacture of thin films.
